(19) United States
(12) Patent Application Publication
(10) Pub. No.: US 2013/0239959 A1
(43) Pub. Date: Sep. 19, 2013

(54) LARYNGEAL MASK AIRWAY DEVICE

(71) Applicant: Archibald Ian Jeremy Brain, Victoria (SC)

(72) Inventor: Archibald Ian Jeremy Brain, Victoria (SC)

(73) Assignee: Laryngeal Mask Company Limited, Victoria, Mahe (SC)

(21) Appl. No.: 13/820,726

(22) PCT Filed: Sep. 6, 2011
(86) PCT No.: PCT/GB2011/001308

§ 371 Date: Jun. 5, 2013

(30) Foreign Application Priority Data
Sep. 6, 2010 (GB) 1014773.4

(51) Int. Cl. A61M 16/04; A61M 25/01

(52) U.S. Cl. CPC A61M 16/0434; A61M 25/01
USPC 128/202.16

(57) ABSTRACT

The invention relates to a laryngeal mask airway device (10) to facilitate lung ventilation in a patient, the device comprising an airway tube (12) extending from a proximal end (12a) to a distal end (12b) and opening, at the distal end, into the interior of a mask portion (14), the mask portion including a backplate (16) and a cuff (18), the mask portion shaped to seal around the circumference of the laryngeal inlet without penetrating into the interior of the larynx, wherein the mask portion of the device includes guiding means (20), said guiding means being adapted such that, when the laryngeal mask airway device is in use with a drug delivery catheter (22), the guiding means guides the drug delivery catheter towards the larynx.

The present invention relates to a laryngeal mask airway device adapted for drug delivery.

The laryngeal mask airway device is a well-known device that is useful for establishing airways in unconscious patients. U.S. Pat. No. 4,509,514 is one of the many publications that describe laryngeal mask airway devices and is a useful source of background information. Such devices have been in use for many years and offer an alternative to the older, even better known, endotracheal tube. For at least seventy years, endotracheal tubes comprising a long slender tube with an inflatable balloon disposed at the tube's distal end have been used for establishing airways in unconscious patients. In operation, the endotracheal tube's distal end is inserted through the mouth of the patient, past the patient's laryngeal inlet (or glottic opening), and into the patient's trachea. Once so positioned, the balloon is inflated so as to form a seal with the interior lining of the trachea. After this seal is established, positive pressure may be applied to the tube's proximal end to ventilate the patient's lungs. Also, the seal between the balloon and the inner lining of the trachea protects the lungs from aspiration (e.g., the seal prevents material regurgitated from the stomach from being aspirated into the patient's lungs).

Although they have been enormously successful, endotracheal tubes suffer from several major disadvantages. The principal disadvantage of the endotracheal tube relates to the difficulty of properly inserting the tube. Inserting an endotracheal tube into a patient is a procedure that requires a high degree of skill, even more so when the patient is a child or and infant. Also, even for skilled practitioners, insertion of an endotracheal tube is sometimes difficult or not possible. Also, inserting an endotracheal tube normally requires manipulations of the patient's head and neck and further requires the patient's jaw to be forcibly opened widely. These necessary manipulations make it difficult, or undesirable, to insert an endotracheal tube into a patient who may be suffering from a neck injury.

In contrast to the endotracheal tube, it is relatively easy to insert a laryngeal mask airway device into a patient and thereby establish an airway. Also, the laryngeal mask airway device is a “forgiving” device in that even if it is inserted improperly, it still tends to establish an airway. Also, the laryngeal mask airway device may be inserted with only relatively minor manipulations of the patient's head, neck, and jaw. Further, the laryngeal mask airway device provides for ventilation of the patient's lungs without requiring contact with the sensitive inner lining of the trachea and the size of the airway established is typically significantly larger than the size of the airway established with an endotracheal tube. Also, the laryngeal mask airway device does not interfere with coughing to the same extent as endotracheal tubes. Largely due to these advantages, the laryngeal mask airway device has enjoyed increasing popularity in recent years.

In the case of respiratory diseases, it is sometimes necessary to deliver drugs directly into the lungs. Respiratory Distress Syndrome (RDS) is an example of such a disease, in which the patient has a deficiency in pulmonary surfactant. Pulmonary surfactant is a substance naturally found in the lungs, which reduces alveolar collapse by decreasing surface tension in the alveoli. This condition affects more particularly newborns and is a major cause of mortality in premature infants. It has been observed that the more premature the infant, the less pulmonary surfactant production occurs because of lung immaturity and the higher the probability for RDS.

Respiratory failure may be avoided by administrating an exogenous surfactant, which may be naturally or synthetically obtained, directly into the pulmonary system. This can for example be achieved by intubating the trachea but this method presents obvious disadvantages for the patient.

Attempts have been made to inject exogenous surfactant through an endotracheal tube. However, due to the difficulty of properly inserting and positioning the tube, the substance is not properly administered to the lungs. It has to be pointed out that a major issue with this treatment is its considerable expense and any loss of exogenous surfactant by improper administration should be avoided. Improper administration of exogenous surfactant may also be harmful to the patient, in particular when the patient is a premature infant. Indeed, if the surfactant is splashed against the vocal cords, it might trigger a laryngeal spasm, which is likely to provoke the cords into closing.

In WO 200807197 there is described a laryngeal mask airway device comprising a conduit adapted to direct a liquid substance through the glottic opening thereby enabling the delivery of liquid drugs directly to the pulmonary system. Nonetheless, some clinicians prefer to deliver liquid drugs using a catheter and so there exists a demand for a laryngeal mask airway device adapted for liquid drug delivery using a catheter.

In accordance with a first aspect of the present invention there is provided a laryngeal mask airway device to facilitate lung ventilation in a patient, the device comprising an airway tube extending from a proximal end to a distal end and opening, at the distal end, into the interior of a mask portion, the mask portion including a backplate and a cuff, the mask portion shaped to seal around the circumference of the laryngeal inlet without penetrating into the interior of the larynx, wherein the mask portion includes guiding means, said guiding means being adapted such that, when the laryngeal mask airway device is in use with a drug delivery catheter, the guiding means guides the drug delivery catheter towards the larynx.

In a first embodiment, the guiding means comprises a moveable surface, which moveable surface can adopt a first state in which the surface lies substantially flat against an interior surface of the hollow mask portion, and a second state in which, when the laryngeal mask airway device is in use with a drug delivery catheter, the moveable surface guides the drug delivery catheter towards the larynx.

Laryngeal mask airway devices made according to this first embodiment may additionally comprise means to move the moveable support surface from the first state to the second state, which means are externally accessible when the laryngeal mask airway device is in use. The means to move the moveable support surface from the first state to the second state may comprise a rod or cord that extends from a proximal end to a distal end. The proximal end of the rod or cord may be externally accessible when the laryngeal mask airway device is in use and acts upon the distal end of the rod or cord, which distal end is attached or hinged to the moveable support surface. This allows a user to move the moveable support surface from the first state to the second state by pushing or pulling the proximal end of the rod or cord.

In a second embodiment, the guiding means comprises an inflatable cushion, which inflatable cushion can adopt a first state in which the inflatable cushion is deflated, and can adopt a second state in which the inflatable cushion is inflated, and wherein, when the laryngeal mask airway device is in use with a drug delivery catheter and the inflatable cushion is inflated, the inflatable cushion guides the drug delivery catheter towards the larynx.

Any means of providing an inflatable cushion to guide the drug delivery catheter towards the larynx may be used with this second embodiment. For example, the inflatable cushion may comprise a shell of flexible material adhered to the interior surface of the backplate. Alternatively, the inflatable cushion may comprise a layer of flexible material adhered to the interior surface of the backplate at its periphery.

Laryngeal mask airway devices made according to this second embodiment may additionally comprise a first flexible tube, which first flexible tube is externally accessible when the laryngeal mask airway device is in use. The first flexible tube may be used to inflate and deflate the inflatable cushion and may further comprise check-valve means to enable a certain inflation or deflation of the inflatable cushion to be maintained.

The cuff of the laryngeal mask airway device may be made of a deformable material or the laryngeal mask airway device may be inflatable. In embodiments in which the cuff of the laryngeal mask airway device is inflatable, the device may comprise a second flexible tube, which second flexible tube is externally accessible when the laryngeal mask airway device is in use. The second flexible tube may be used to inflate and deflate the inflatable cuff and may further comprise check-valve means to enable a certain inflation or deflation of the inflatable cuff to be maintained.

In certain embodiments of the present invention, the laryngeal mask airway device may comprise a gastric drain tube. When the laryngeal mask airway device is in use, the gastric drain tube extends from a distal end located proximate the distal end of the mask to a proximal end located outside of the mouth of a patient. The gastric drain tube enables enable active or passive removal of gastric discharge from the upper oesophageal sphincter.

In accordance with a second aspect of the present invention a kit is provided, wherein the kit comprises a laryngeal mask airway device according to the present invention and a drug delivery catheter.

In accordance with a third aspect of the present invention, a method for delivering a liquid substance to a patient's larynx, without the liquid substance contacting the patient's vocal chords, is provided. The method comprises:
a). inserting a laryngeal mask airway device according to the present invention into a patient's airway;
b). inserting a drug delivery catheter through the laryngeal mask airway device;
c). causing the guiding means of the laryngeal mask airway device to adopt the second state such that the drug delivery catheter is guided towards the larynx; and
d). dispensing a liquid substance using the drug delivery catheter.

The method may include the use of viewing means to assist with guiding of the catheter.

The invention will further be described by way of example with reference to the accompanying drawings in which the same reference numerals are used to indicate the same or similar parts wherein:

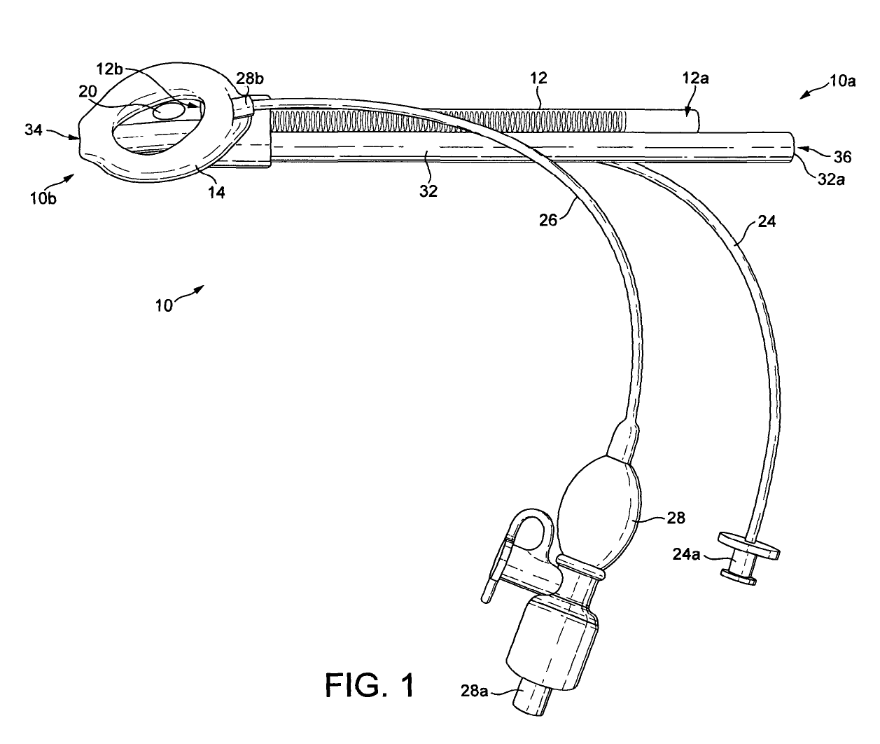

FIG. 1 illustrates a ventral view of a laryngeal mask airway device constructed according to the present invention;

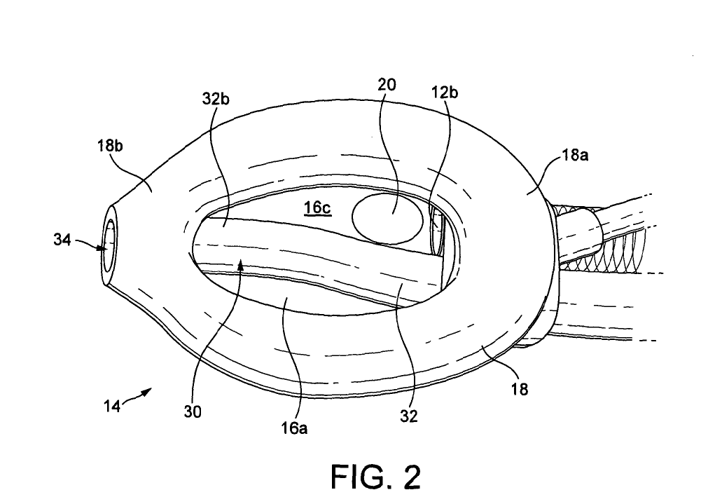

FIG. 2 illustrates an enlarged view of the mask portion of the laryngeal mask airway device of FIG. 1;

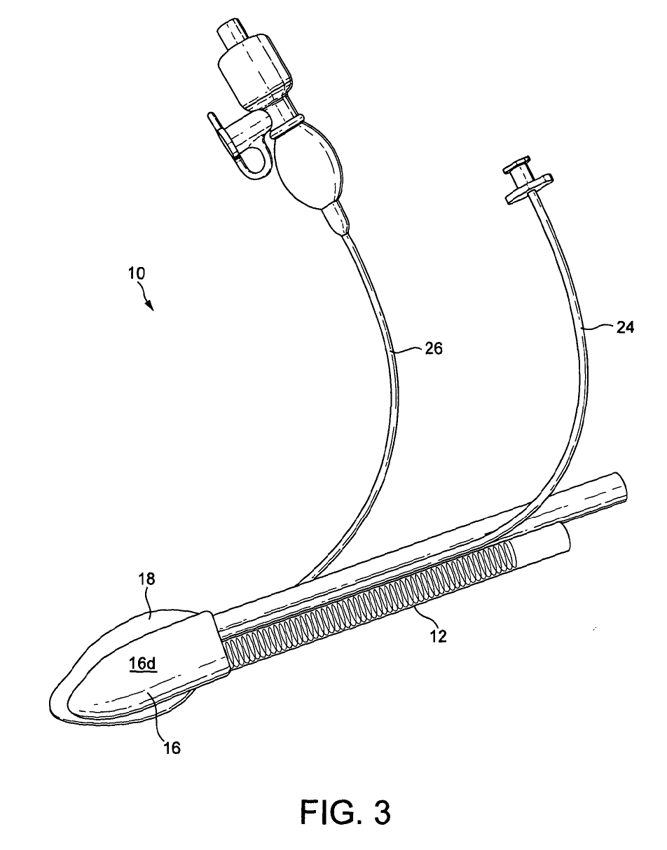

FIG. 3 illustrates a dorsal view of the laryngeal mask airway device of FIG. 1;

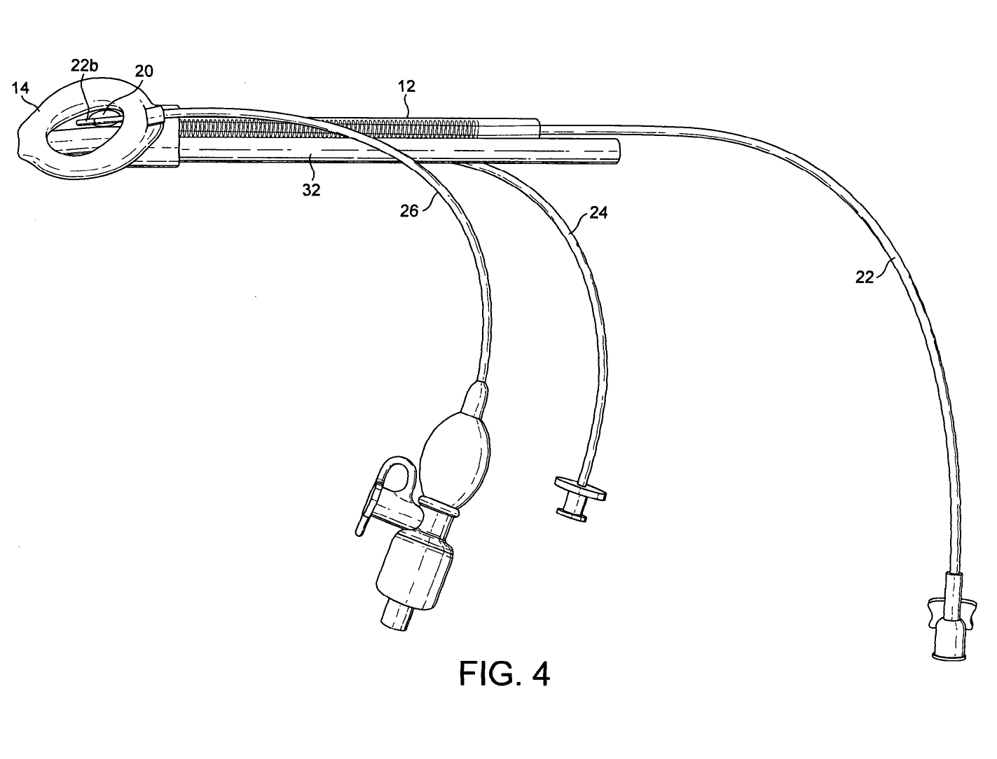

FIG. 4 illustrates a ventral view of the laryngeal mask airway device of FIG. 1 with a drug delivery catheter inserted into the device;

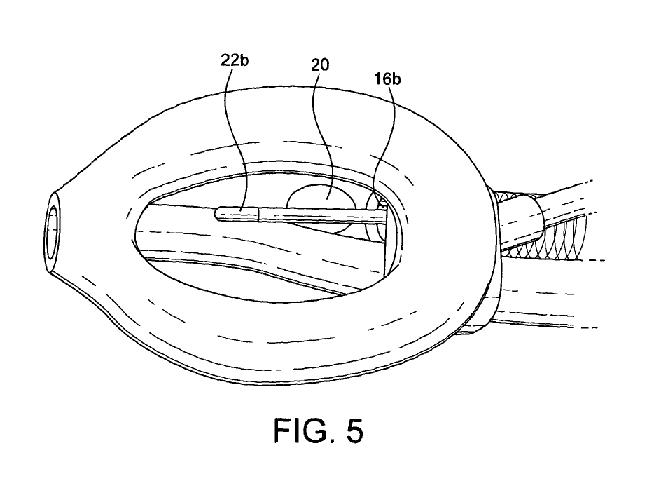

FIG. 5 illustrates an enlarged view of the portion mask of the laryngeal mask airway device of FIG. 1 with a drug delivery catheter inserted into the device;

FIG. 6 illustrates an enlarged view of the mask portion of the laryngeal mask airway device of FIG. 1 with a drug delivery catheter inserted further into the device;

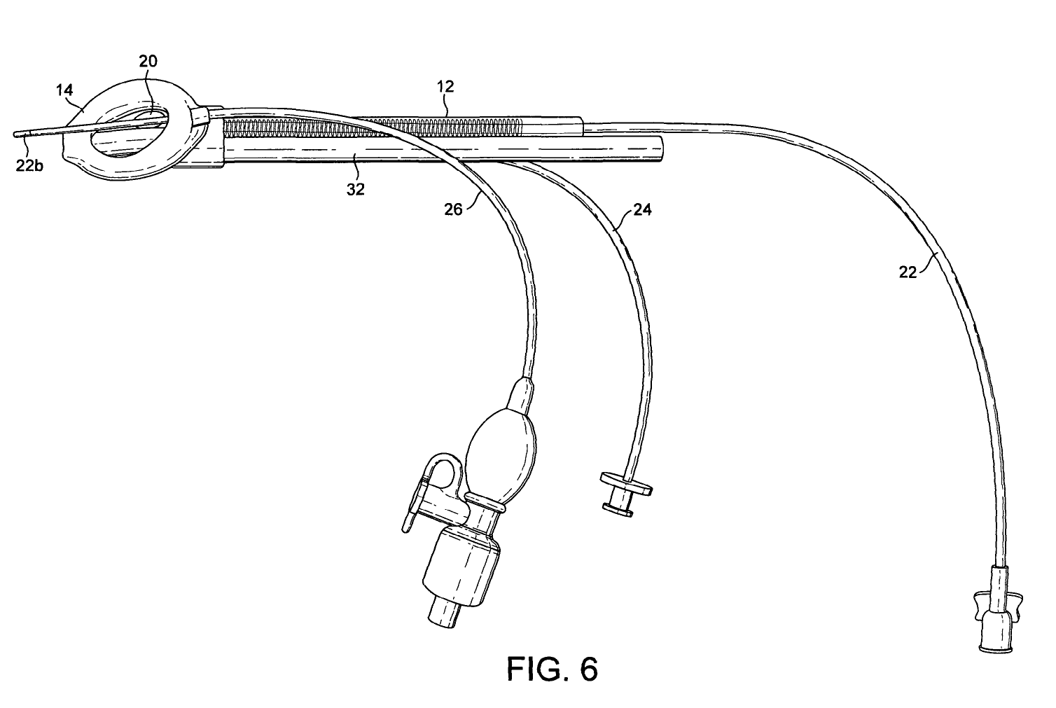

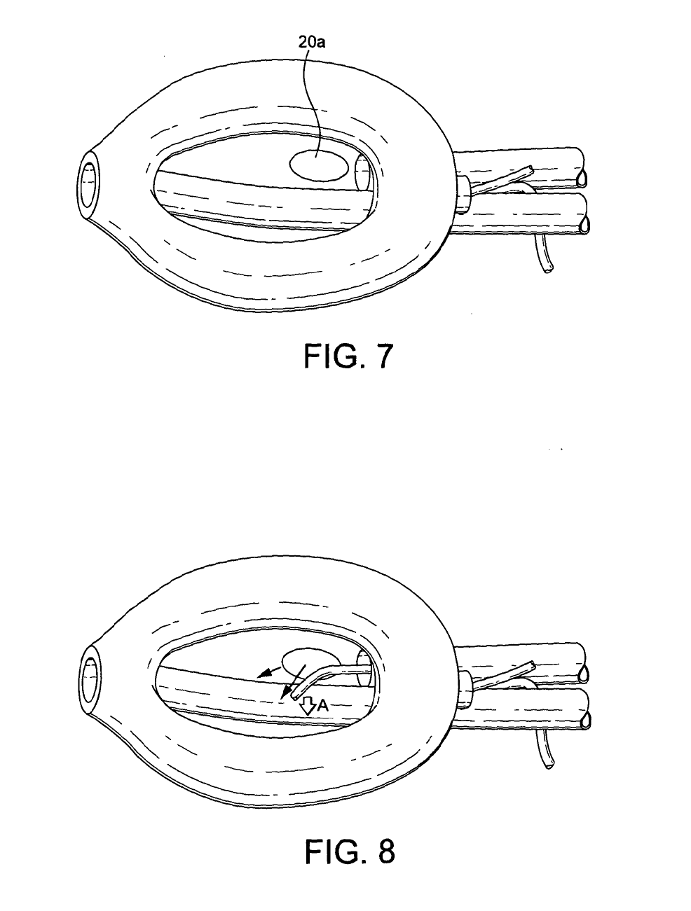

FIG. 7 illustrates an enlarged view of the mask portion of a device according to the invention in a first position;

FIG. 8 illustrates an enlarged view of the mask portion of the device of FIG. 7 in a second position;

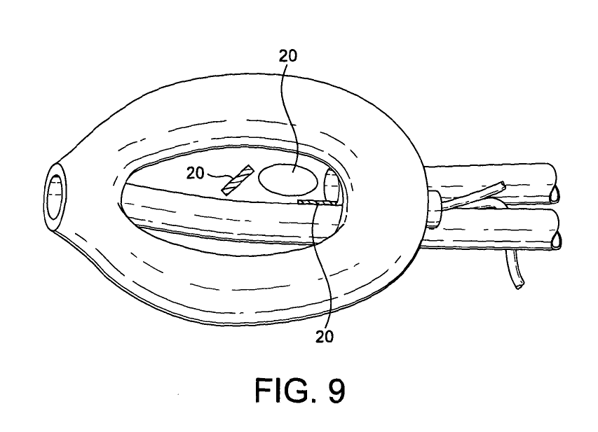

FIG. 9 illustrates an enlarged view of the mask portion of an alternative embodiment of device according to the invention;

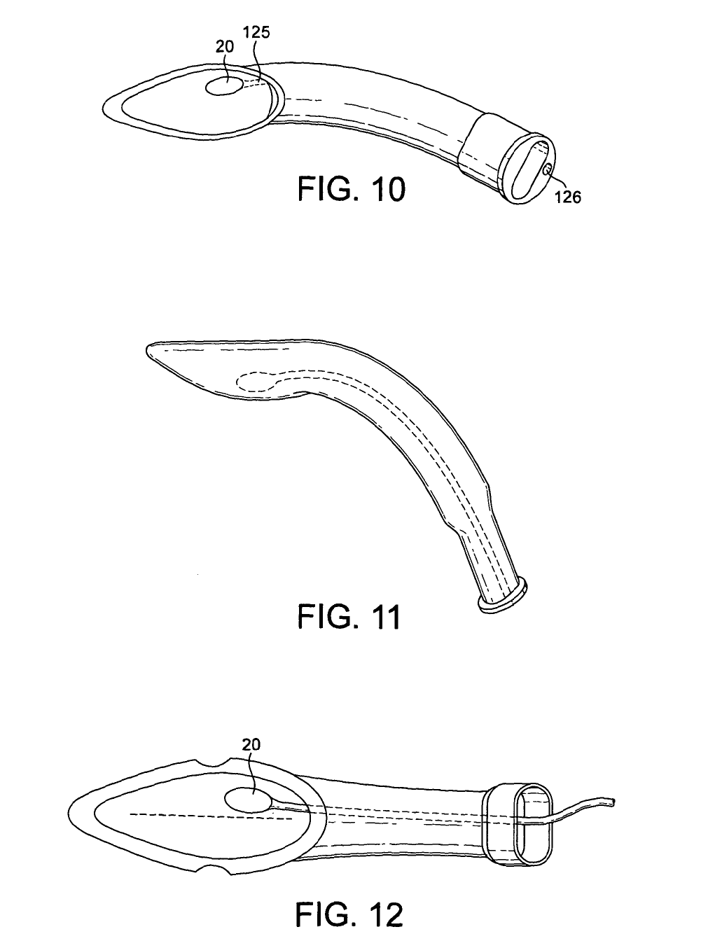

FIG. 10 illustrates a ventral view of a part of a device according to the invention;

FIG. 11 illustrates a dorsal three-quarter perspective view of the part illustrated in FIG. 10; and

FIG. 12 illustrates a ventral view of a second embodiment of the part illustrated in FIG. 10.

Referring to the drawings, there is illustrated a laryngeal mask airway device 10 to facilitate lung ventilation in a patient, the device comprising an airway tube 12 extending from a proximal end 12a to a distal end 12b and opening, at the distal end, into the interior of a mask portion 14, the mask portion including a backplate 16 and a cuff 18, the mask portion shaped to seal around the circumference of the laryngeal inlet without penetrating into the interior of the larynx, wherein the mask portion of the device includes guiding means 20, said guiding means being adapted such that, when the laryngeal mask airway device is in use with a drug delivery catheter 22, the guiding means guides the drug delivery catheter towards the larynx.

The laryngeal mask airway device 10 of the present invention is similar to prior art laryngeal mask airway devices, in that it consists of the basic parts which make up most, if not all, such devices, i.e. an airway tube 12 and a mask portion 14. With reference to the Figures, the device 10 has a proximal end 10a (the end nearest the user when the device is in use), a distal end 10b (the end farthest from the user when the device is in use), a dorsal or pharyngeal side, and a ventral or laryngeal side.

The airway tube 12, extends from a proximal end 12a to a distal end 12b, and the distal end 12b opens into the interior of the hollow mask portion 14. The airway tube 12 may be resiliently deformable or relatively rigid, to enable it to assist in insertion of the device 10 into a patient, acting as a handle and a guide. The airway tube 12 may be made of any material that is currently used for such purposes as will be apparent to one of skill in the art, for example, silicone rubber or plastics materials. It may be straight and flexible or molded into an appropriately anatomically-curved shape. Alternatively, the airway tube 12 may be made of a relatively rigid sterilisable material, for example stainless steel.

The mask portion 14 includes a backplate 16 and a cuff 18, extending from a proximal end 18a to a distal end 18b (FIG. 2). The backplate 16 in this embodiment comprises a bowl 16a that includes an aperture 16b that is in fluid communication with the airway tube 12. The bowl has a laryngeal, or ventral surface 16c (FIG. 2) and a pharyngeal or dorsal surface 16d (FIG. 3). The cuff 18 is, in this embodiment, inflatable, although it need not be as long as it is capable of fitting within the space behind the patient's larynx and forming a seal around the circumference of the laryngeal inlet without the device 10 penetrating into the interior of the larynx. Device 10 also includes a cuff inflation tube 26 for inflating the inflatable cuff 18, which extends from a distal end 28b that is coupled to the proximal end 18a of cuff 18 to a distal end 28b that is located outside of the patient when the device 10 is in use. A check valve 28 is located within the cuff inflation tube 26. This is a structure that is common to most prior art laryngeal mask airway devices as will be appreciated by one of skill in the art.

In contrast to prior art laryngeal mask airway devices, the device 10 according to the invention includes guiding means 20, the purpose of which is to assist in the correct disposition of a drug delivery catheter 22 placed in the device for the purpose of delivering a drug such as a surfactant to the pulmonary system. The guiding means 20 may take the form of a fixed wall or upstand that is disposed on the ventral surface 16c of the bowl 16a adjacent the aperture 16b so as to be contacted by the tip 22b of a drug delivery catheter 22 as it is advanced into the mask portion via the airway tube. Examples of possible dispositions of guiding means 20 are illustrated schematically in FIG. 9.

Although the present invention contemplates a fixed guiding means 20, it has been found surprisingly that some degree of control over the position of the tip 22b of the drug delivery catheter 22 can also help avoid problems associated with prior art devices. Although such a construction may increase the complexity of manufacture of devices and may make sterilisation of reusable devices more difficult, the ability to finely adjust the position of the catheter tip relative to the mask body when the device is in situ in the patient gives the user a degree of control over the precise direction of drug delivery that would not have been thought necessary, particularly in the smaller paediatric sizes of device. Furthermore, the ability to move the guiding means 20 away from the aperture of the airway tube ensures that a fully open and effective airway without obstruction is provided when the airway tube is not being used for dosing via a catheter. Thus a moveable guiding means 20 provides particular and unexpectedly valuable advantages.

In this example the guiding means 20 takes the form of an inflatable deflector. As can be seen in FIGS. 4, 5, 7 and 8, the inflatable deflector comprises a skin 20a that is fixed around its edge in fluid-tight sealing relation to ventral surface 16c of the bowl in the manner of a balloon or blister. It is fixed adjacent to the aperture 16b that communicates with the airway tube. Fixation may be achieved with appropriate adhesive or any suitable means depending on the materials of the skin 20a and the back plate 16, known to one of skill in the art. The skin may be thrilled from any suitable material such as silicone or a plastics material and can be resiliently deformable. When a non-resiliently deformable material is selected it is sized sufficiently so that it assumes an appropriate size on inflation.

Device 10 further includes a first flexible tube 24 for selectively inflating and deflating the inflatable deflector 20. As will be appreciated by the skilled worker various options exist for attachment of the inflation tube 24 to the deflector. In the embodiment illustrated in FIGS. 1 to 9, the flexible tube 24 passes through an aperture in the backplate 16 from the dorsal to the ventral side where it terminates within the deflector. On the dorsal side the flexible tube 24 runs to proximal end 24a that is located outside of the patient when the device 10 is in use. FIGS. 10 and 11 illustrate an alternative embodiment in which the airway tube and backplate 16 are formed as a single molding. In these figures the cuff 18 is omitted for clarity. Thus it can be seen that in this embodiment the integrally molded airway tube and backplate includes a channel 125 formed in the dorsal wall of the airway tube and running from the deflector to a port 126 in the wall at its proximal end. In a yet further alternative embodiment, which is again an integrally molded backplate and airway tube construction the deflector inflation tube 24 is formed on the ventral surface of the back plate and passes along the interior of the airway tube exiting at its proximal end. In all cases, a one-way valve may be provided to prevent unwanted deflation of the deflector, and where the skin of the deflector is formed from a resiliently deformable material, simple means such as a bellows can be provided at the proximal end to enable a user to increase the pressure within the deflector by applying manual force thereto.

In the embodiment illustrated in the Figures, device 10 also includes an oesophageal drain 30, the drain 30 comprising a conduit 32 extending from an inlet 34 at the distal end 32b of the conduit to an outlet 36 at the proximal end 32a of the conduit 32. The proximal end 32a of the conduit 32 is located outside of the patient when the device is in use

In use, the device 10 is inserted through the patient's mouth and down through the throat past the epiglottis until the mask 14 comes to rest with the distal end of the cuff 18b in the base of the throat, lying against the upper end of the normally closed oesophagus (which the mask 14 cannot easily enter because of its dimensions). The cuff 18 is then inflated to seal around the inlet to the larynx. Referring particularly to FIGS. 7 and 8, a drug delivery catheter 22 may then be inserted through the airway tube 12 of the device 10 until the distal end of the drug delivery catheter 22b is located at or on the guide means 20 (see FIG. 8). The guide means 20 is then inflated and the distal end of the drug delivery catheter 22b is deflected towards the centre of the glottic opening of the patient's airway in the direction of arrow ‘A’. The drug delivery catheter 22 may then be inserted further through the device 10 (see FIG. 6) before a liquid substance is dispensed using the drug delivery catheter 22. As will be clear to the skilled worker, the extent of inflation of the guide means 20 can be varied by the user to exert control over the extent of deflection of the tip 22b.

The guide means 20 is advantageous in that it provides a means of accurately dispensing a liquid substance into the pulmonary system using a drug delivery catheter 22, without requiring any further invasive procedure. Thus, a safe and accurate delivery of a liquid substance can be achieved using devices according to the invention. In the context of the present invention, “liquid substance” means a substance essentially in a liquid or fluid state, and may therefore include substances in the form of emulsions, dispersions and the like. Thus, it has been demonstrated that the present invention provides a device that enables the safe and accurate administration of a liquid drug substance to a patient, through the glottis to the pulmonary system.

1. A laryngeal mask airway device to facilitate lung ventilation in a patient, comprising an airway tube extending from a proximal end to a distal end and opening, at the distal end, into the interior of a mask portion, the mask portion including a backplate and a cuff, the mask portion shaped to seal around the circumference of the laryngeal inlet without penetrating into the interior of the larynx, wherein the device comprises guiding means located within the interior of the mask portion, said guiding means being adapted such that, when the laryngeal mask airway device is in use with a drug delivery catheter, the guiding means guides the drug delivery catheter towards the larynx.

2. The device according to claim 1, wherein the guiding means comprises a moveable support surface, which moveable support surface can adopt a first state in which the moveable support surface lies flat against the interior surface of the mask portion, and can adopt a second state in which, when the laryngeal mask airway device is in use with a drug delivery catheter, the moveable support surface guides the drug delivery catheter towards the larynx.

3. The device according to claim 2, wherein the laryngeal mask airway device comprises means to move the moveable support surface from the first state to the second state, which means are externally accessible when the laryngeal mask airway device is in use.

4. The device according to claim 1, wherein the guiding means comprises an inflatable deflector, which inflatable deflector can adopt a first state in which the inflatable deflector is deflated, and can adopt a second state in which the inflatable deflector is inflated, and wherein, when the laryngeal mask airway device is in use with a drug delivery catheter and the inflatable deflector is inflated, the inflatable deflector guides the drug delivery catheter towards the larynx.

5. The device according to claim 4, wherein the inflatable deflector comprises a skin of flexible material adhered to the interior surface of the backplate.

6. The device according to claim 4, wherein the inflatable deflector comprises a layer of flexible material and the periphery of the inflatable material is adhered to the interior surface of the backplate.

7. The device according to claim 4, wherein the device comprises a first flexible tube for use in supplying air to the inflatable deflector, which first flexible tube is externally accessible when the laryngeal mask airway device is in use.

8. The device according to claim 1, wherein the cuff is inflatable.

9. The device according to claim 1, wherein the device comprises a gastric drain tube, said gastric drain tube extending, when in use, from a distal end located proximate the distal end of the mask to a proximal end located outside of the mouth of a patient.

10. A kit comprising a laryngeal mask airway device according to claim 1, and a drug delivery catheter.

11. A method for delivering a liquid substance to a patient's larynx, without the liquid substance contacting the patient's vocal chords, the method comprising:
a). inserting a laryngeal mask airway device according to claim 1 into the patient's airway;
b). inserting a drug delivery catheter through the laryngeal mask airway device;
c). causing the guiding means of the laryngeal mask airway device to adopt the second state such that the drug delivery catheter is guided towards the larynx; and
d). dispensing a liquid substance using the drug delivery catheter.
